(19) United States
(12) Patent Application Publication
(10) Pub. No.: US 2023/0398165 A1
(43) Pub. Date: Dec. 14, 2023

(54) COMPOSITE FIBER SPONGE CONTAINING SULFORAPHANE

(71) Applicant: Taipei Veterans General Hospital, Taipei City (TW)

(72) Inventors: Shu-Chen LIAO, Taipei City (TW); Li-Yin CHIEN, Taipei City (TW); Jin-Lain MING, Taipei City (TW); Li-Hwa LIN, Taipei City (TW)

(21) Appl. No.: 18/333,283

(22) Filed: Jun. 12, 2023

(30) Foreign Application Priority Data
Jun. 13, 2022 (TW) 111121802

(51) Int. Cl. A61K 36/31; A61K 47/38; A61K 47/32; A61K 9/70; D01F 2/08; D01F 2/28; D01F 9/04

(52) U.S. Cl. CPC A61K 36/31; A61K 47/38; A61K 47/32; A61K 9/703; D01F 2/08; D01F 2/28; D01F 9/04

(57) ABSTRACT

A composite fiber sponge containing sulforaphane is produced by the step of subjecting a composite liquid to a molding treatment and a crosslinking treatment. The composite liquid contains a composite fiber including an extract of a Brassicaceae plant, a polysaccharide, and a carboxymethyl cellulose fiber. The extract of the Brassicaceae plant contains the sulforaphane. A composite fiber material containing sulforaphane is also disclosed.

### CROSS-REFERENCE TO RELATED APPLICATION

This application claims priority to Taiwanese Invention Patent Application No. 111121802, filed on Jun. 13, 2022.

### FIELD

The disclosure relates to a composite fiber sponge and a composite fiber material containing sulforaphane prepared from the same.

### BACKGROUND

Sulforaphane, which exists in Brassicaceae plants (e.g., cauliflower, cabbage, broccoli, bok choy, and Chinese cabbage), is a product obtained via hydrolysis of glucosinolate utilizing myrosinase, and is reported to have various beneficial effects on human body, including anti-oxidant, anti-inflammatory and anti-cancer properties, and is also capable of eliminating swelling and reducing stasis.

Since sulforaphane confers the aforesaid advantages effects on the human body, there is a need for those skilled in the art to develop a long-acting sulforaphane-containing product which can continuously release sulforaphane so as to meet the needs and demands of industry.

### SUMMARY

Accordingly, in a first aspect, the present disclosure provides a composite fiber sponge containing sulforaphane, which can alleviate at least one of the drawbacks of the prior art. The composite fiber sponge is produced by the step of:
subjecting a composite liquid to a molding treatment and a crosslinking treatment,
wherein the composite liquid contains a composite fiber including an extract of a Brassicaceae plant, a polysaccharide, and a carboxymethyl cellulose fiber, the extract of the Brassicaceae plant containing the sulforaphane.

In a second aspect, the present disclosure provides a composite fiber material containing sulforaphane, which can alleviate at least one of the drawbacks of the prior art. The composite fiber material includes:
a substrate which is selected from the group consisting of the aforesaid composite fiber sponge and a nonwoven fabric; and
a functional layer disposed on the substrate, the functional layer being formed by electrospinning a mixture onto a surface of the substrate, the mixture containing an extract of a Brassicaceae plant containing sulforaphane, and polyvinyl alcohol.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

By conducting research, the applicant surprisingly found that a composite fiber sponge or a composite fiber material made from a polysaccharide-based composite fiber, which includes an extract of a Brassicaceae plant containing sulforaphane, can slowly release the sulforaphane, and hence is expected to be capable of exhibiting the pharmacological effect of the sulforaphane over an extended period of time.

Accordingly, the present disclosure provides a composite fiber sponge containing sulforaphane, which is produced by the step of:
subjecting a composite liquid to a molding treatment and a crosslinking treatment,
wherein the composite liquid contains a composite fiber including an extract of a Brassicaceae plant, a polysaccharide, and a carboxymethyl cellulose fiber, the extract of the Brassicaceae plant containing the sulforaphane.

In certain embodiments, the composite fiber including the extract of the Brassicaceae plant may be made from a fibrous stock solution containing the extract of the Brassicaceae plant and a polysaccharide.

In certain embodiments, the extract of the Brassicaceae plant may be produced by hydrolyzing a material of a Brassicaceae plant containing glucosinolate utilizing an extract of a Brassicaceae plant containing myrosinase.

According to the present disclosure, the extract of the Brassicaceae plant containing myrosinase suitable for use in this disclosure is not particularly limited, and may be prepared using techniques well-known to those skilled in the art (for example, see Wang Jian-Dong et al. (2003), Food and Fermentation Industries, Volume 29, Issue 2).

It should be understood that the procedures and operating conditions for extracting the extract of the Brassicaceae plant containing myrosinase may be adjusted according to practical requirements, and are within the expertise and routine skills of those skilled in the art.

In certain embodiments, the Brassicaceae plant may be selected from the group consisting of a cabbage, a broccoli, a cauliflower, a Brussels sprout, a turnip, a mustard green, a bok choy, a Chinese cabbage, and combinations thereof. In an exemplary embodiment, the Brassicaceae plant is a cabbage. In another exemplary embodiment, the Brassicaceae plant is a broccoli.

According to the present disclosure, the Brassicaceae plant may be a fresh material that is not processed, or may be obtained through a processing treatment selected from the group consisting of a drying treatment, a grinding treatment, a chopping treatment, a comminuting treatment, a solid-liquid separation treatment, and combinations thereof.

According to the present disclosure, the extract of the Brassicaceae plant containing myrosinase may be prepared using an extraction solvent. Examples of the extraction solvent may include, but are not limited to, distilled water, acetone, ammonium sulfate, ethanol, ethyl acetate, and methylene dichloride. In an exemplary embodiment, the extraction solvent is acetone.

According to the present disclosure, a weight ratio of the Brassicaceae plant to the extraction solvent may range from 1:0.25 to 1:9. In an exemplary embodiment, the weight ratio of the Brassicaceae plant to the extraction solvent is 1:1.

In certain embodiments, the polysaccharide may be selected from the group consisting of an alginate, a chitosan, and a combination thereof.

In certain embodiments, the alginate is a salt of an alginic acid formed by crosslinking of the alginic acid with a divalent metal ion or a polyvalent metal ion, and examples of the alginate may include, but are not limited to, calcium alginate, magnesium alginate, zinc alginate, copper alginate, barium alginate, and iron alginate. In an exemplary embodiment, the alginate is calcium alginate.

According to the present disclosure, the crosslinking treatment, which is performed after the molding treatment, may be carried out by adding the composite liquid to an aqueous solution containing calcium ions (Ca 2+). Examples of the aqueous solution may be a calcium chloride aqueous solution and a calcium hydroxide aqueous solution. In certain embodiments, the aqueous solution may be the calcium chloride aqueous solution having a concentration ranging from 0.1 g/mL to 2 g/mL. In an exemplary embodiment, the aqueous solution is the calcium chloride aqueous solution having a concentration of 1 g/mL.

According to the present disclosure, the crosslinking treatment may also be carried out by adding the composite liquid to an alkaline solution. Examples of the alkaline solution may be a Tris(hydroxymethyl)aminomethane buffer solution, a sodium carbonate solution, a sodium bicarbonate solution, an ammonium solution, a sodium hydroxide solution, and a potassium hydroxide solution. In certain embodiments, the alkaline solution may be the Tris(hydroxymethyl)aminomethane buffer solution having a concentration ranging from 0.5 g/mL to 2 g/mL. In an exemplary embodiment, the alkaline solution is the Tris(hydroxymethyl)aminomethane buffer solution having a concentration of 1 g/mL.

In certain embodiments, the crosslinking treatment may be carried out at a stirring rate ranging from 1000 rpm to 8000 rpm. In an exemplary embodiment, the stirring rate is 4000 rpm.

The present disclosure also provides a composite fiber material containing sulforaphane, which includes:
a substrate which is selected from the group consisting of the aforesaid composite fiber sponge and a nonwoven fabric; and
a functional layer disposed on the substrate, the functional layer being formed by electrospinning a mixture onto a surface of the substrate, the mixture containing an extract of a Brassicaceae plant containing sulforaphane, and polyvinyl alcohol (PVA).

As used herein, the terms “electrospinning” and “electrostatic spinning” may be interchangeably used. According to the present disclosure, techniques of electrospinning are within the expertise and routine skills of those skilled in the art. In certain embodiments, the electrospinning may be performed under a working voltage ranging from 5 kV to 60 kV and a working distance ranging from 5 cm to 60 cm. In certain embodiments, the electrospinning may be performed under a working voltage ranging from 15 kV to 20 kV and a working distance of 20 cm.

According to the present disclosure, the substrate may be made into various sizes and shapes using techniques well known to those skilled in the art. Examples of the shapes may include, but are not limited to, a spherical shape, an ellipsoidal shape, a cylindrical shape, a square shape, a rectangle shape, and a polygonal shape. In an exemplary embodiment, the substrate is made into a square shape.

According to the present disclosure, the functional layer may further include a polymer material. Examples of the polymer material may include, but are not limited to, a natural polymer material (e.g., collagen, chitosan, and silk protein), a synthetic polymer material (e.g., polyvinyl alcohol, polyethylene glycol, polyacrylonitrile, polylactic acid, polylactic acid-glycolic acid copolymer, polyimide, nylon, polycaprolactone, and polyurethane), and a combination thereof. In an exemplary embodiment, the polymer material is polyvinyl alcohol.

In certain embodiments, a weight ratio of the polymer material to the substrate may range from 1:1 to 1:100. In certain embodiments, the weight ratio of the polymer material to the substrate may range from 1:4 to 1:20. According to the present disclosure, the nonwoven fabric may be obtained as commercial products, or may be prepared using techniques that are within the expertise and routine skills of those skilled in the art (for example, see Nonwoven Fabric: Manufacturing and Applications (2009), Photochemical Industry, Volume 31, Issue 4).

According to the present disclosure, the nonwoven fabric may be made from a material selected from the group consisting of a carboxymethyl cellulose (CMC) fiber, a calcium alginate fiber, a rayon fiber, a polyester fiber (e.g., a polypropylene fiber), a wood pulp fiber, a cotton fiber, a bacterial cellulose fiber, a silk fiber, and combinations thereof. In certain embodiments, the nonwoven fabric may be made from a CMC fiber and a sodium alginate fiber having a weight ratio ranging from 1:0.11 to 1:9. In an exemplary embodiment, the weight ratio of the CMC fiber and the sodium alginate fiber is 1:1.

According to the present disclosure, the nonwoven fabric may have a basis weight ranging from 20 g/m² to 1000 g/m 2. In certain embodiments, the nonwoven fabric may have a basis weight ranging from 100 g/m² to 200 g/m 2.

The disclosure will be further described by way of the following examples. However, it should be understood that the following examples are solely intended for the purpose of illustration and should not be construed as limiting the disclosure in practice.

### EXAMPLES

### General Experimental Materials

1. The cabbage (Brassica oleracea var. capitata) and the broccoli (Brassica oleracea var. italica) used in the following examples were purchased from Taipei Agricultural Products Marketing Co., Ltd. during spring season.
2. The alginate (Cat. No. 20130305044) and the chitosan (Cat. No. BF054, with a degree of deacetylation greater than 90%) used in the following examples were purchased from Emperor Chemical Co., Ltd.
3. Carboxymethyl cellulose (CMC) fiber:

The CMC fibers (with a degree of substitution ranging from 0.2 to used in the following examples were prepared by the methods with reference to WO 1993/012275 A1. Briefly, Tencel™ (Lyocell or Modal cellulosic fibers) (with diameters ranging from 10 μm to 20 μm and lengths ranging from 3 cm to 8 cm) were immersed in an alkaline solution (containing a 40% sodium hydroxide solution and a 95% ethanol solution mixed in a volume ratio of 2:3) for 2 hours. Afterward, the alkaline solution was replaced with a chloroacetic acid solution to perform etherification for 20 hours, thereby obtaining sodium carboxymethyl cellulose (CMC-Na) fibers. Later on, the CMC-Na fibers were washed with an ethanol solution having a concentration ranging from 70% to 95%, followed by drying in an oven at 65° C. for 48 hours, thereby obtaining the CMC fibers.

### General Experimental Procedures

1. High Performance Liquid Chromatography (HPLC) Analysis:

The concentration of sulforaphane in a respective one of the test samples of the following examples was determined by HPLC analysis using a high-performance liquid chromatography system (Waters, Model: 600) and an ultraviolet (UV) detector (Waters, Model: 486), which was carried out according to the methods described in Han D. et al. (2011), Int. J. Mol. Sci., 12(3):1854-1861. The operating parameters and conditions for performing HPLC analysis were shown in Table 1 below.

TABLE 1

| Type of chromatography column | C18 column | |
|---|---|---|
| | (Shodex ™, Model: 4E) | |
| Size of chromatography column | 4.6 mm × 250 mm | |
| Temperature of chromatography | 30° | C. |
| column | | |
| Test sample injection volume | 10 | μL |
| Measuring wavelength | 205 | nm |

| | |
|---|---|
| Mobile phase | Acetonitrile/water |
| | (20:80, v/v) |
| Flow rate of test sample | 1.0 (mL/min) |

2. Preparation of Chitosan Solution:

An appropriate amount of the chitosan was added to a 0.1 M acetic acid solution so as to obtain a chitosan solution having a concentration of 1% (w/v).

### Example 1 Preparation of Extract of Brassicaceae Plant Containing Sulforaphane

1. Preparation of Extract of Cabbage Containing Myrosinase:

First, an appropriate amount of fresh cabbage was subjected to homogenization so as to form a homogenized solution, which was then subjected to filtration with a double layer of gauze, so as to obtain a filtrate. Next, the filtrate was mixed with acetone in a weight ratio of 1:1 at 5° C., followed by centrifugation at a temperature of 4° C. to 6° C. and a speed of 4000 rpm for 10 minutes, so as to obtain a pellet (i.e., an extract of cabbage containing myrosinase). The resultant pellet was collected, and then subjected to lyophilization, thereby obtaining a dried powder of the extract of cabbage containing myrosinase.

2. Preparation of Extract of Cabbage Containing Sulforaphane:

An appropriate amount of cabbage was subjected to lyophilization and pulverization, and the resultant dried powder was then mixed with water in a weight ratio of 1:2 to form a mixture, followed by adjusting the pH value of the mixture to be within the range of pH 4 to pH 4.5 using lactic acid.

Afterward, an appropriate amount of the dried powder of the extract of cabbage containing myrosinase obtained in Section 1 of this example was added to the mixture having a pH value ranging from 4.0 to 4.5, thus allowing a hydrolysis reaction to proceed at a temperature ranging from 30° C. to 35° C. for 2 to 3 hours, so as to form a hydrolyzed product. The hydrolyzed product was then subjected to lyophilization, so as to obtain a dried powder of the hydrolyzed product.

Subsequently, the dried powder of the hydrolyzed product was mixed with 95% ethanol in a weight ratio of 1:5 and left standing for 3 hours, followed by filtration with a qualitative filter paper (ADVANTEC®, No. 2), so as to obtain a filtrate and a residue. The residue was then mixed with 95% ethanol in a weight ratio of 1:3 and left standing for 3 hours, followed by filtration with a qualitative filter paper (ADVANTEC®, No. 2), so as to obtain another filtrate and another residue. Later on, each of the filtrates obtained above was collected, and then concentrated at a temperature ranging from 35° C. to 40° C. under reduced pressure so as to remove the ethanol therein, thereby obtaining the extract of cabbage containing sulforaphane in paste form (hereinafter referred to as “cabbage extract”).

3. Preparation of Extract of Broccoli Containing Sulforaphane:

The procedures for preparing the extract of broccoli containing sulforaphane (hereinafter referred to as “broccoli extract”) were similar to those of the cabbage extract as described in Sections 1 and 2 of this example, except that the broccoli was used instead of the cabbage.

4. Determination of Concentration of Sulforaphane:

The concentration of sulforaphane contained in each of the cabbage extract and the broccoli extract was determined according to the methods described in Section 1 of the General Experimental Procedures. The results showed that the sulforaphane concentration determined in each of the cabbage extract and the broccoli extract was 0.35±0.059 mg/g and 2.203±0.098 mg/g, respectively.

### Example 2 Preparation of Composite Fiber Sponge According to the Present Disclosure

1. Preparation of Calcium Alginate-Based Composite Fiber Containing the Cabbage Extract:

First, an appropriate amount of the cabbage extract obtained in Section 2 of Example 1 was dissolved in dimethyl sulfoxide (DMSO) to form a mixture having a concentration of the cabbage extract of 1 g/mL. Next, the mixture was mixed with a 1 g/mL aqueous solution of sodium alginate in a weight ratio of 1:9 to form a mixed solution, followed by adding the mixed solution in a dropwise manner to a 1% by weight solution of calcium chloride under stirring at 4000 rpm, so as to allow a crosslinking reaction to proceed, where exchange of calcium ions with sodium ions may take place, thereby obtaining a calcium alginate-based composite fiber containing the cabbage extract (hereinafter referred to as “composite fiber A”).

2. Preparation of Calcium Alginate-Based Composite Fiber Sponge Containing the Cabbage Extract:

A 2 g/mL aqueous solution of the CMC fibers, a 2 g/mL aqueous solution of sodium alginate, the cabbage extract obtained in Section 2 of Example 1, and the composite fiber A obtained in Section 1 of this example were mixed with a distinct ratio to prepare a composite liquid of each of AS1 to AS5 as shown in Table 2 below.

TABLE 2

| | Amount by weight (%) | | | | |
|---|---|---|---|---|---|
| Composite | CMC | Sodium | Cabbage | Composite | |
| liquid | fibers | alginate | extract | fiber A | |
| AS1 | 1.0 | 1.0 | — | — | The |
| AS2 | 1.0 | 1.0 | 0.01 | — | balance |
| AS3 | 1.0 | 0.9 | 0.01 | 0.1 | being |
| AS4 | 1.0 | 0.8 | 0.02 | 0.2 | water |
| AS5 | 1.0 | 0.5 | 0.05 | 0.5 | |

Afterward, the composite liquid of each of AS1 to AS5 was placed into a mold to be subjected to a molding treatment using lyophilization, so as to form a shaped material. Next, the shaped material was added into a 1% by weight solution of calcium chloride to be subjected to a crosslinking treatment for 1 to 3 minutes, followed by lyophilization, thereby obtaining a calcium alginate-based composite fiber sponge. The components and the amounts thereof in the calcium alginate-based composite fiber sponge of each of AS1 to AS5 were shown in Table 3 below.

TABLE 3

| Calcium | | | | | |
|---|---|---|---|---|---|
| alginate-based | Amount by weight (%) | | | | |
| composite fiber | CMC | Sodium | Cabbage | Composite | Calcium |
| sponge | fibers | alginate | extract | fiber A | chloride |
| AS1 | 49.5 | 49.5 | — | — | 1.0 |
| AS2 | 49.25 | 49.25 | 0.5 | — | |
| AS3 | 49.5 | 44.5 | 0.5 | 5 | |
| AS4 | 49.5 | 39.5 | 1.0 | 10 | |
| AS5 | 49.5 | 24.5 | 2.5 | 25 | |

3. Preparation of Chitosan-Based Composite Fiber Sponge Containing the Cabbage Extract:

The procedures for preparing the chitosan-based composite fiber sponge containing the cabbage extract were similar to those of the calcium alginate-based composite fiber sponge containing the cabbage extract as described in Sections 1 and 2 of this example, except that for preparing the chitosan-based composite fiber sponge containing the cabbage extract, 1 g/mL solution of chitosan was used instead of the 1 g/mL aqueous solution of sodium alginate, and the 1 g/mL Tris(hydroxymethyl)aminomethane buffer solution was used instead of the 1% by weight solution of calcium chloride to allow an acid-base neutralization reaction to proceed. Moreover, instead of obtaining the calcium alginate-based composite fiber containing the cabbage extract gained in Section 1, i.e., the composite fiber A, here, the chitosan-based composite fiber containing the cabbage extract (hereinafter referred to as “composite fiber C”) was obtained. The components and the amounts thereof in the chitosan-based composite fiber sponge of each of CS1 to CS4 were shown in Table 4 below.

TABLE 4

| Chitosan- | | | | | |
|---|---|---|---|---|---|
| based | Amount by weight (%) | | | | |
| composite | CMC | | Cabbage | Composite | Calcium |
| fiber sponge | fibers | Chitosan | extract | fiber C | chloride |
| CS1 | 49.25 | 49.25 | 0.5 | — | 1.0 |
| CS2 | 49.5 | 44.5 | 0.5 | 5 | |
| CS3 | 49.5 | 39.5 | 1.0 | 10 | |
| CS4 | 49.5 | 24.5 | 2.5 | 25 | |

### Example 3 Preparation of Composite Fiber Material According to the Present Disclosure

1. Preparation of Electrospun Polyvinyl Alcohol (PVA) Composite Fiber Sponge:

First, PVA and the cabbage extract obtained in Section 2 of Example 1 were respectively dissolved in DMSO so as to form a mixture having 1.2% by weight of the cabbage extract and 12% by weight of the PVA. Next, each of different amounts of the mixture was subjected to electrospinning (under a working voltage ranging from 15 kV to 20 kV and a working distance of 20 cm), so as to form a PVA-based composite fiber (hereinafter referred to as “composite fiber P”) (serving as a functional layer of the composite fiber material) onto a surface of the calcium alginate-based composite fiber sponge of AS1 obtained in Section 2 of Example 2 (serving as a substrate of the composite fiber material), thereby obtaining an electrospun polyvinyl alcohol (PVA) composite fiber sponge. The components and the amounts thereof in the electrospun PVA composite fiber sponge of each of MNS1 to MNS3 were shown in Table 5 below.

TABLE 5

| Electrospun | Amount by weight (%) | | | | |
|---|---|---|---|---|---|
| PVA composite | CMC | Sodium | Cabbage | Composite | Calcium |
| fiber sponge | fibers | alginate | extract | fiber P | chloride |
| MNS1 | 47.0 | 47.0 | 0.05 | 5 | 1.0 |
| MNS2 | 44.5 | 44.5 | 1.00 | 10 | |
| MNS3 | 37.0 | 37.0 | 2.50 | 25 | |

2. Preparation of Electrospun PVA Composite Fiber Nonwoven:

The calcium alginate fiber used in the following experiments was prepared according to the methods described in EP 3660191 4A. Briefly, a 3% to 5% solution of sodium alginate serving as a spinning solution was extruded into a coagulation bath containing 5% of calcium chloride to be subjected to wet spinning, followed by drafting, boarding, washing and drying treatments performed in sequence, thereby obtaining the calcium alginate fiber.

Afterward, the CMC fibers and the calcium alginate fiber were mixed in a weight ratio of 1:1 so as to obtain a mixed fiber. Next, the mixed fiber was formed into a thin web using a carding machine, followed by cross-lapping the thin web so that a thick multi-layered web was formed. Afterward, a needle punching machine was employed to permit the thick multi-layered web to be formed into a nonwoven fabric having a basis weight ranging from 100 g/m² to 200 g/m 2.

The procedures for preparing the electrospun PVA composite fiber nonwoven were similar to those of the electrospun PVA composite fiber sponge described in Section 1 of this example, except that in preparation of the electrospun PVA composite fiber nonwoven, the nonwoven fabric was used instead of the calcium alginate-based composite fiber sponge of AS1. The components and the amounts thereof in the electrospun PVA composite fiber nonwoven of each of MNW1 to MNW3 were shown in Table 6 below.

TABLE 6

Amount by weight (%)

| Electrospun | | Calcium | | |
|---|---|---|---|---|
| PVA composite | CMC | alginate | Cabbage | Composite |
| fiber nonwoven | fibers | fiber | extract | fiber P |
| MNW1 | 47.5 | 47.5 | 0.05 | 5 |
| MNW2 | 45.0 | 45.0 | 1.00 | 10 |
| MNW3 | 37.5 | 37.5 | 2.50 | 25 |

### Example 4 Evaluation of the Efficacy of the Composite Fiber Sponge and the Composite Fiber Material According to the Present Disclosure in Releasing Sulforaphane

### Experimental Procedures

A respective one of the composite fiber sponges AS2 to AS5 and CS1 to CS4 obtained in Example 2 and the composite fiber materials MNS1 to MNS3 and MNW1 to MNW3 obtained in Example 3 was cut into a piece having a size of 1 cm×1 cm with a weight of approximately 0.2 g. Next, each piece was immersed in 10 mL of a saline solution at 32° C. for 120 hours. At each of the predetermined time points of 1^{st}, 3^{rd}, 6^{th}, 16^{th}, 24^{th}, 48^{th}, 72^{nd}, and 96^{th} hour after the start of immersion, 0.1 mL of the saline solution was taken out to serve as a test sample. Determination of the concentration of sulforaphane contained in the test sample was carried out according to the methods described in Section 1 of the General Experimental Procedures, so as to calculate the amount of sulforaphane released from the respective composite fiber sponge and composite fiber material.

The release rate of sulforaphane of each of the composite fiber sponges and the composite fiber materials at the predetermined time point was calculated using the following Equation:

A=(B/C)×100
where A=release rate of sulforaphane (%)
   B=amount of sulforaphane released at the predetermined time point (mg)
   C=total amount of sulforaphane in composite fiber sponge or composite fiber material (mg) (calculated based on each preparation process)

Result:

Referring to Table 7, at the 6th hour after the start of immersion, the release rates of sulforaphane of both the calcium alginate-based composite fiber sponge AS2 and the chitosan-based composite fiber sponge CS1 had already surpassed 97%, while the release rates of sulforaphane of all the calcium alginate-based composite fiber sponges AS3 to AS5 and the chitosan-based composite fiber sponges CS2 to CS4 had yet reached 40%, where the release rates of sulforaphane of the chitosan-based composite fiber sponges CS2 to CS4 were even less than 25%, indicating that the composite fiber sponges according to the present disclosure (i.e., which contain either the composite fiber A or the composite fiber C) have noticeably excellent slow-release effect of sulforaphane.

Each of the composite fiber materials, i.e., the electrospun PVA composite fiber sponges of MNS1 to MNS3 and the electrospun PVA composite fiber nonwovens of MNW1 to MNW3, also showed an excellent slow-release effect of sulforaphane as those of the composite fiber sponges.

TABLE 7

| | | Release rate of sulforaphane (%) | | | | | | | | |
|---|---|---|---|---|---|---|---|---|---|---|
| | | Pre-determined time point (hour) | | | | | | | | |
| | | 1^{st} | 3^{rd} | 6^{th} | 16^{th} | 24^{th} | 48^{th} | 72^{nd} | 96^{th} | 120^{th} |
| Composite | AS2 | 63.7 | 92.4 | 98.7 | 98.9 | 99.1 | — | — | — | — |
| fiber | AS3 | 13.2 | 21.3 | 30.2 | 38.6 | 41.5 | 76.2 | 85.4 | 90.4 | 96.7 |
| sponge | AS4 | 15.7 | 23.6 | 35.1 | 40.3 | 45.2 | 78.4 | 88.2 | 92.1 | 98.1 |
| | AS5 | 20.6 | 30.1 | 36.9 | 42.1 | 50.2 | 80.6 | 90.6 | 95.2 | 97.8 |
| | CS1 | 52.5 | 78.3 | 97.8 | 98.7 | 98.7 | — | — | — | — |
| | CS2 | 4.3 | 10.3 | 15.6 | 28.4 | 34.9 | 50.1 | 63.2 | 80.1 | 90.9 |
| | CS3 | 5.2 | 14.1 | 20.2 | 30.2 | 37.9 | 53.2 | 66.6 | 84.6 | 93.2 |
| | CS4 | 6.1 | 16.5 | 24.3 | 31.4 | 42.5 | 55.9 | 72.4 | 88.5 | 94.5 |
| Electrospun | MNS1 | 21.5 | 28.5 | 37.4 | 48.3 | 62.4 | 82.5 | 94.6 | 98.4 | — |
| PVA | | | | | | | | | | |
| composite | MNS2 | 24.3 | 33.1 | 42.6 | 53.1 | 69.3 | 85.1 | 97.2 | 98.3 | — |
| fiber | | | | | | | | | | |
| sponge | MNS3 | 30.2 | 36.9 | 47.2 | 30.2 | 70.1 | 88.5 | 96.6 | 97.7 | — |
| Electrospun | MNW1 | 20.3 | 26.5 | 34.6 | 50.6 | 53.2 | 79.9 | 94.6 | 97.6 | — |
| PVA | | | | | | | | | | |
| composite | MNW1 | 25.9 | 32.9 | 15.6 | 28.4 | 64.9 | 84.3 | 97.6 | 98.3 | — |
| fiber | | | | | | | | | | |
| nonwoven | MNW1 | 28.6 | 37.8 | 47.2 | 30.2 | 67.9 | 93.2 | 98.6 | 97.6 | — |

In sum, the composite fiber sponge and the composite fiber material according to the present disclosure, each of which is made from a polysaccharide-based composite fiber that includes an extract of a Brassicaceae plant containing sulforaphane, can exhibit the effect of sustained release of sulforaphane, and hence is capable of extending the duration of pharmaceutical activity of sulforaphane.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A composite fiber sponge containing sulforaphane produced by the step of:
subjecting a composite liquid to a molding treatment and a crosslinking treatment,
wherein the composite liquid contains a composite fiber including an extract of a Brassicaceae plant, a polysaccharide, and a carboxymethyl cellulose fiber, the extract of the Brassicaceae plant containing the sulforaphane.

2. The composite fiber sponge as claimed in claim 1, wherein the composite fiber including the extract of the Brassicaceae plant is made from a fibrous stock solution containing the extract of the Brassicaceae plant and a polysaccharide.

3. The composite fiber sponge as claimed in claim 2, wherein the extract of the Brassicaceae plant is produced by hydrolyzing a material of a Brassicaceae plant containing glucosinolate utilizing an extract of a Brassicaceae plant containing myrosinase.

4. The composite fiber sponge as claimed in claim 1, wherein the Brassicaceae plant is selected from the group consisting of a cabbage, a broccoli, a cauliflower, a Brussels sprout, a turnip, a mustard green, a bok choy, a Chinese cabbage, and combinations thereof.

5. The composite fiber sponge as claimed in claim 3, wherein the extract of the Brassicaceae plant containing myrosinase is obtained from a Brassicaceae plant selected from the group consisting of a cabbage, a broccoli, a cauliflower, a Brussels sprout, a turnip, a mustard green, a bok choy, a Chinese cabbage, and combinations thereof.

6. The composite fiber sponge as claimed in claim 1, wherein the polysaccharide is selected from the group consisting of an alginate, a chitosan, and a combination thereof.

7. The composite fiber sponge as claimed in claim 2, wherein the polysaccharide of the fibrous stock solution is selected from the group consisting of an alginate, a chitosan, and a combination thereof.

8. A composite fiber material containing sulforaphane, comprising:
a substrate which is selected from the group consisting of a composite fiber sponge as claimed in claim 1 and a nonwoven fabric; and
a functional layer disposed on the substrate, the functional layer being formed by electrospinning a mixture onto a surface of the substrate, the mixture containing an extract of a Brassicaceae plant containing sulforaphane, and polyvinyl alcohol.

9. The composite fiber material as claimed in claim 8, wherein the nonwoven fabric is made from a material selected from the group consisting of a carboxymethyl cellulose (CMC) fiber, a calcium alginate fiber, a rayon fiber, a polyester fiber, a wood pulp fiber, a cotton fiber, a bacterial cellulose fiber, a silk fiber, and combinations thereof.

10. The composite fiber material as claimed in claim 9, wherein the nonwoven fabric is made from a carboxymethyl cellulose fiber and a calcium alginate fiber.
